(19) United States
(12) Patent Application Publication
(10) Pub. No.: US 2005/0002877 A1
(43) Pub. Date: Jan. 6, 2005

(54) One and only toe nail/finger nail fungus killer

(76) Inventor: Brett Kenneth Miller, Atwater, CA (US)

Correspondence Address: Brett Miller, U.S.P. Atwater, P.O. Box 019001, Atwater, CA, 95301, US

(21) Appl. No.: 10/361,203

(22) Filed: Jul. 3, 2003

(51) Int. Cl. A61K 33/42; A61K 31/716; A61K 31/045; A61K 33/14

(52) U.S. Cl. 424/61; 424/601; 514/57; 514/738; 424/676

(57) ABSTRACT

This new art is medicine that kills (cures) thick ugly toe/finger nail fungus, (or any kind of nail fungus) and the major difference with this medicine, is that this medicine is applied to the external body using a combination of sodium monofluorophosphate, sodium fluoride, and a cream/gum base. After this medicine is applied, the toe/finger nail is covered with a plastic/rubber “toe/finger housing” for 24 hours a day, and any type of nail fungus will be dead in 6 to 10 days. This “toe/finger housing” also works with other medicine.

### BACKGROUND OF THE INVENTION

The subject matter of the claimed invention is: killing (curing) thick ugly toe/finger nail fungus by using medicine on the external body.

The specific problems of the prior art is that there was no external body medicine that cured thick ugly toe/finger nail fungus, and the internal medicine that does exist can cause damage to a persons internal organs.

### BRIEF SUMMARY OF THE INVENTION

The object of this invention is to kill/cure thick ugly toe/finger nail fungus by applying medicine to the external body, (toe/finger nail) which also is the advantage of this invention, because there is no existing medicine that can kill/cure thick ugly nail fungus when applied to the external body, until now. The only other external nail fungus medication that exist, is basically a gimmick that does not kill/cure thick nail fungus, it can only be applied to mild nail fungus and it takes about 6 months or more of use, and only has about a 8% cure rate. This new medicine so far has a 100% cure rate, and the user can see a healthy fungus free nail starting to grow in about 3 weeks to a month. This new medication should only take about 6 to 10 days to kill the thick ugly nail fungus

The following is the substance or general idea of the claimed invention: File off and cut off as much of the toe/finger nail fungus as possible, then a solution of sodium monofluorophosphate, sodium fluoride, sorbitol, cellulose gum, and water is applied to the nail fungus area and also applied all around the nail, especially near the cuticle, then the nail is covered with a plastic/rubber “toe/finger housing” for 24 hours a day, and any type of nail fungus will be dead in 6 to 10 days.

### DETAILED DESCRIPTION OF THE INVENTION

The chemical solution used in this new invention is made by mixing 1% sodium monofluorophosphate, 0.10% sodium fluoride, and adding it to a solution of sorbitol and cellulose gum and water, (henceforth sodium monofluor. sol.) that when mixed makes a medium moist paste that will spread easily.

The medical device used to cover the chemical solution and the toe/finger nail fungus is called a “toe/finger housing” and it can be made in 2 different ways, the first is by placing a snug fitting rubber balloon over the toe/finger nail, and the second way is to use a 4 inch by 7 inch plastic/rubber type sheet (that is thin like a common house hold trash bag) which will be tied on.

The toe/finger nail must be cleared of the diseased fungus part of the nail (as much as possible, emphasis added) using a good file and clippers. Once the toe/finger nail is prepared, apply about a pea size amount of the sodium monofluor. sol. to the diseased toe/finger nail, cover the nail area, (even if the nail has been filed off) and cover the skin near the nail edges. MOST IMPORTANT cover the entire cuticle and cover the skin area up to ½ inch back from the cuticle.

Now it is time to cover the nail and the sodium monofluor. sol. that has been applied to the diseased nail. If the balloon style “toe/finger housing” is prefered for use, then simply pull the balloon style toe//finger housing over the toe/finger, cover enough of the toe/finger so that the balloon wont come off.

When you use the plastic//rubber wrap style toe/finger housing, wrap the wrap around the toe/finger, wrap it around over and over until all the wrap is used. If the finger is being wrapped, wrap it 2 inches down the finger, so that there is an excess of 2 inches of plastic wrap sticking out past the tip of the finger, fold the excess plastic back over the toe/finger so that it is laying on top of the toe/finger. Now take a piece of tape and wrap it snug around the plastic wrap, so that it holds the fold in place and keeps the plastic wrap from sliding off the toe/finger.

Change the medicine and toe/finger housing every 12 hours. Apply this process 24 hours a day for 6 to 10 days and the thick nail fungus will be dead and a new nail will grow. This medicine will soak in and kill the fungus.

1.) What I claim as my invention is: The chemical solution (medicine) that kills thick ugly toe/finger nail fungus or any type of fungus, which is applied to the external body, (toe/finger nail) and which is made from the following chemicals: sodium monofluorophosphte, sodium fluoride, sorbitol, cellulose gum, and water. I also claim the process of using this medicine with the “toe/finger housing” invention mentioned in the second claim below. No person has ever claimed this chemical solution as a toe/finger nail fungus killer. No person has ever claimed the process of using this chemical solution.

2.) What I claim as my invention is: The “toe/finger housing,” which covers the medicine that is mentioned in the first claim above, and covers the diseased nail that has thick ugly fungus or any type of fungus. This “toe/finger housing” directly assist killing toe/finger nail fungus. There are 2 different types of “toe/finger housings,” just as mentioned in the specification, there is the balloon style and the wrap style. No person has ever claimed this type of medical device for the purpose of killing toe/finger nail fungus. No person has ever claimed the process of using this medical device (toe/finger housing). This medical device can work with other fungus medicine.
